(19) United States
(12) Patent Application Publication
(10) Pub. No.: US 2021/0375452 A1
(43) Pub. Date: Dec. 2, 2021

(54) Method and System for Remote Augmented Reality Medical Examination

(71) Applicants: Abe Lin, Los Angeles, CA (US); James Tai, Los Angeles, CA (US)

(72) Inventors: Abe Lin, Los Angeles, CA (US); James Tai, Los Angeles, CA (US)

(21) Appl. No.: 17/331,623

(22) Filed: May 26, 2021

Related U.S. Application Data
(60) Provisional application No. 63/120,925, filed on Dec. 3, 2020.
(60) Provisional application No. 63/120,691, filed on Dec. 2, 2020.
(60) Provisional application No. 63/120,687, filed on Dec. 2, 2020.
(60) Provisional application No. 63/030,281, filed on May 26, 2020.

(51) Int. Cl. G16H 40/67; G16H 80/00; G06F 3/01; G06K 9/00

(52) U.S. Cl. CPC G16H 40/67; G16H 80/00; G06K 9/00355; G06K 9/00671; G06K 9/00362; G06F 3/017

(57) ABSTRACT

A system for remote examination of patient by a doctor is disclosed, the system comprising: a doctor mobile computer device comprising a first set of processors, the first set of processors executing an application embedded within the doctor mobile computer device; a patient mobile computer device comprising a second set of processors, the second set of processors executing the application embedded within the patient mobile computer device; the application comprising: a camera module wherein the module captures a video feed of an area of a body part of the patient and sends the video feed to a patient viewing module and a doctor view module to display the area of body part; the patient viewing module displaying the video feed; the doctor viewing module displaying the video feed and a plurality of inspection gesture buttons comprising a one-finger palpation gesture button, a two-finger palpation gesture button, a hand grabbing gesture button, a posterior palpation gesture button and a plurality of unique gesture graphics corresponding to the inspection gesture buttons;
a control module wherein the control module allows the doctor to select one of the inspection gesture buttons and one of the unique gesture graphics superimpose onto the area of the body part of the video feed wherein the control module further allows the doctor to move the unique gesture graphic within the area of the video feed as the unique gesture graphic continues to superimpose onto the area of the body part.

### INCORPORATION BY REFERENCE

This application claims the benefit of priority under 35 U.S.C. 119(e) to the filing date of U.S. Provisional application No. 63/120,925, entitled “Administrator's Real-Time Training of a Video-Based Machine Learning, Medical Examination and Diagnosis Apparatus,” which was filed on Dec. 3, 2020, and U.S. Provisional Application No. 63/120,691, entitled “Detection of Centers of Rotation for Video Medical Examination and Diagnosis using Machine Learning,” which was filed on Dec. 2, 2020, and U.S. Provisional Application No. 63/120,687, entitled “Automated, AR Video Medical Examination and Diagnosis using Machine Learning,” which was filed on Dec. 2, 2020, and U.S. Provisional Application No. 63/030,281, entitled “Augmented Reality Virtual Examination,” which was filed on May 26, 2020, and which are incorporated herein by reference in their entirety.

### FIELD OF THE INVENTION

The present invention relates to telemedicine systems and methods. Specifically, it relates to a virtual physical examination platform that utilizes augmented reality and machine vision technologies during a live video examination session.

### BACKGROUND OF THE INVENTION

In the traditional setting, astute physicians start the physical examination as the patient walks into the room because this provides immediate clues about patients' characteristics. Observing the patient's gait and level of effort to walk into the room and to sit up on the examination table can be useful to assess his or her functional performance. Although eliciting the patient's description of a health issue can be done and should be done as foundational activities of a physical exam, good physicians usually verify the patient's description with their own observation of or a physical examination of the patient.

Modern technologies enable virtual examinations that include telephonic and video examinations. However, telephonic examinations have the limitation of inhibiting the ability of the physician to visualize the user's body part of interest during the examination. Video examinations allow an physician to visualize the user's anatomy of interest and presents an advantage over telephonic examinations. However, even though the elicitation and observation can be done over telephonic and video conferences, both methods lack the palpation capability.

Currently, in order to navigate through differential diagnoses, a video examination could allow a clever physician to achieve indirect palpation if the physician directs the patient verbally to go through various anatomic examination steps and, as such, gets the patient to palpate different anatomic landmarks his or herself, and to report back the sensations. During an virtual examination, the patient can point to an area, but oftentimes, the physician needs to pinpoint a specific area or navigate to other anatomic landmarks in order to rule out other diagnoses. The possible complexity of the examination and the imprecise communication with an untrained patient can make the examination session inefficient and frustrating for both the physician and patient, and can reduce both the physician's and patient's confidence in the video examination and the accuracy of the diagnosis. Worse, without some degree of confirmation and certainty, responsible physicians would hesitate to recommend a course of treatment because if the determination is wrong, the physicians lose credibility and, perhaps, cause serious mistreatment of the patient's disease.

Because the importance of a complete medical diagnosis for the subsequent successful treatment, and patient confidence in the course of treatment, the accuracy of the physicians' medical determination is very important and widely acknowledged. However, during a deadly pandemic, because of the need to keep vulnerable patients, and physicians, at home and away from health care facilities to decrease risk of virus transmission, physicians have adapted to using a virtual examination system, and, thus, uses of virtual, or telemedicine, visits have rapidly increased. Moreover, beyond a pandemic, telemedicine will remain a viable option because the benefit of providing medical care without moving patients or physicians. Patients can avoid the expense and time involved in driving long distances for medical treatment, or possibly be treated at home. This can help patients avoid taking time off work or schedule a visit more quickly than in a doctor's office.

For the foregoing benefits and drawbacks, there is a great advantage to leverage the augmented reality and machine vision technologies in order to allow a physician to quickly and accurately direct the patient to self-exam, minimizing any potential miscommunication and misdiagnosis. This technology leverage would allow a physician to intuitively and efficiently guide a patient through palpation, percussion, and overall examination in a live video-based examination.

### OBJECT OF THE INVENTION

Accordingly, it is an object of this invention to provide an innovative system and method for a virtual physical examination.

It is an objective of the invention to provide allow a patient and physician to interact over a video visit using multimedia capable devices.

It is another objective of the invention to provide an innovative system and method that allows a physician and patient to engage in a video examination during which the physician directs the patient in various physical examination techniques.

It is an objective of the invention to provide a novel system and method for a virtual physical examination that allows a physician to direct a virtual hand onto the display of a patient in real time in order to accurately guide a physical self-examination.

It is an objective of the invention to provide a novel system and method that allow a physician and patient to see each other on video screens.

It is an objective of the invention to provide a novel system and method that allow a physician to see the patient in a “selfie” mode or normal video mode.

It is an objective of the invention to provide a novel system and method that allow a physician to control an augmented reality overlay that is superimposed on the video of the patient in normal video or “selfie” mode.

It is an objective of the invention to provide a novel system and method that allow a physician to control an augmented reality overlay that has any shape, such as a hand, tool, arrow, or device.

It is an objective of the invention to provide a novel system and method that allow a physician to remotely control an augmented reality overlay on a patient's device by moving their finger over their own mobile, tablet, or electronic device.

It is an objective of the invention to provide a novel system and method that allow a physician to select or change an augmented reality overlay depending on what physical examination maneuver she or he wants to perform, anytime during a virtual physical examination.

It is an objective of the invention to provide a novel system and method that allow a physician changes an overlay forth and back between translucent and solid.

It is an objective of the invention to provide a novel system and method that allow a physician to direct a patient to place their hand, a medical device, or other objects over the overlay in a superimposing position as the overlay guides the hand through examination steps.

It is an objective of the invention to provide a novel system and method that allow a patient to see an overlay over their body and to position the overlay such that it superimposes their own hand, medical device, or other objects.

It is an objective of the invention to provide a novel system and method that allow a physician to display and control more than one overlay.

It is an objective of the invention to provide a novel system and method that allow a physician to move an overlay over various anatomic landmarks sequentially to allow physical examination of different anatomic landmarks.

It is an objective of the invention to provide a novel system and method that allow a digital device to provide feedback data to a digital device.

It is an objective of the invention to provide a novel system and method that allow synchronized multiple screens showing various angles of an area of concern.

It is an objective of the invention to provide a novel system and method that allow a patient to capture images of an area of concern during a virtual physical examination.

It is an objective of the invention to provide a novel system and method that allow a patient to record a self-examination session and forward the multimedia video clip to a physician.

Similarly, it is an objective of the invention to provide a novel system and method that allow a physician to record an instructional multimedia video clip and forward it to a patient so that the patient can self exam.

It is an objective of the current invention to provide a novel system and method that reduce or eliminate the need of moving the patient or physician for a medical consultation, and, thus, the costs in terms of energy and time for such office visits and the risk of transmitting communicable diseases.

It is an objective of the current invention to provide a novel system and method that reduces diagnostic errors associated with virtual examination, unnecessary visits, and encourages physicians to use more virtual visits, and improves patient confidence and satisfaction.

It is an objective of the current invention to provide a novel system and method that save physicians' and patients' time and energy, and, thus, reduce costs of unnecessary overhead and staffing for in-person visits.

### SUMMARY OF INVENTION

In one aspect of the invention, a system for remote examination of patient by a doctor is disclosed, the system comprising: a doctor mobile computer device comprising a first set of processors, the first set of processors executing an application embedded within the doctor mobile computer device; a patient mobile computer device comprising a second set of processors, the second set of processors executing the application embedded within the patient mobile computer device; the application comprising: a camera module wherein the module captures a video feed of an area of a body part of the patient and sends the video feed to a patient viewing module and a doctor view module to display the area of body part; the patient viewing module displaying the video feed; the doctor viewing module displaying the video feed and a plurality of inspection gesture buttons comprising a one-finger palpation gesture button, a two-finger palpation gesture button, a hand grabbing gesture button, a posterior palpation gesture button and a plurality of unique gesture graphics corresponding to the inspection gesture buttons; a control module wherein the control module allows the doctor to select one of the inspection gesture buttons and one of the unique gesture graphics superimpose onto the area of the body part of the video feed wherein the control module further allows the doctor to move the unique gesture graphic within the area of the video feed as the unique gesture graphic continues to superimpose onto the area of the body part.

In one embodiment, the plurality of inspection gesture buttons further comprising a twisting gesture button. In one embodiment the plurality of inspection gesture buttons further comprising a pulling gesture button. In one embodiment the plurality of inspection gesture buttons further comprising a turning gesture button. In one embodiment the plurality of inspection gesture buttons further comprising a pushing up gesture button. In one embodiment the plurality of inspection gesture buttons further comprising a pushing down gesture button. In one embodiment, the plurality of inspection gesture buttons further comprising a two-handed maneuvers gesture button. In one embodiment the plurality of inspection gesture buttons further comprising a two-handed maneuvers gesture button. In one embodiment the plurality of inspection gesture buttons further comprising a 2 hands Lachman's gesture button. In one embodiment, the plurality of inspection gesture buttons further comprising a grabbing gesture button.

In another aspect of the invention, a system for remote examination of patient by a doctor is disclosed, the system comprising: a doctor mobile computer device comprising a first set of processors, the first set of processors executing an application embedded within the doctor mobile computer device; a patient mobile computer device comprising a second set of processors, the second set of processors executing the application embedded within the patient mobile computer device; the application comprising: a camera module wherein the module captures a video feed of an area of a body part of the patient and sends the video feed to a patient viewing module and a doctor view module to display the area of body part; the patient viewing module displaying the video feed; the doctor viewing module displaying the video feed and a examination icon comprising a first body part overlay and a second part body part and a first goniometer overlay and a second goniometer overlay wherein the first body part overlay encloses the first goniometer overlay and the second body part overlay encloses the second goniometer overlay such that when first body part moves in relation to the second body part, the first goniometer overlay moves in relation to the second goniometer overlay and an angular degree display measuring the angular degree between the first goniometer overlay and the second goniometer overlay; a control module wherein the control module superimposes the examination icon onto the area of the body part of the video feed wherein the control module further allows the doctor to move the examination icon within the area of the video feed as the examination icon continues to superimpose onto the area of the body part.

In one embodiment, the control module further allows the doctor to move the first body part overlay in relation to second body part overlay as the first body part overlay, the second body part overlay, the first goniometer overlay, the second goniometer overlay, and the angular degree display continue to superimpose onto the area of the body part.

### BRIEF DESCRIPTION OF THE DRAWINGS

These and other features and advantages of the invention will not be described with reference to the drawings of certain preferred embodiments, which are intended to illustrate and not to limit the invention, and in which

FIG. 1 is a flowchart of an exemplary virtual examination;

FIG. 2 illustrates an embodiment of the current invention that allows users of the system, a physician or patient, to sign in and initiate a virtual examination session;

FIG. 3 illustrates an embodiment of the current invention that can show a remote image of an area of concern pointed at by a hand overlay during a virtual examination;

FIG. 4 illustrates another embodiment of the current invention that can show picture-in-picture livestream of a consulting physician or a patient during a virtual examination;

FIG. 5 illustrates an embodiment of the current invention that can show a patient, on his or her display, a hand overlay representing the consulting physician's hand during a virtual examination;

FIGS. 6A, 6B, and 6C illustrate an embodiment of the current invention during a virtual examination where the physician uses hand and goniometer overlays to guide the patient to bend his hand and wrist by a 89 degrees angle.

FIGS. 7A and 7B illustrate an embodiment of the current invention during a virtual examination where the physician uses arm and goniometer overlays to guide the patient to bend his arm by a 90 degrees angle.

FIG. 8 illustrates an embodiment of the current invention that comprises mobile devices connected over a wireless LAN or broadband network;

FIG. 9 illustrate an embodiment of the current invention during a virtual examination where the physician uses overlays to guide the patient to perform palpation.

FIG. 10 illustrate an embodiment of the current invention during a virtual examination where the physician uses overlays to guide the patient to perform palpation.

FIG. 11 illustrates another embodiment of the current invention during a virtual examination where the physician uses hand and goniometer overlays to guide the patient to bend his hand and wrist in which the entire body and head of patient appears in the screen.

FIG. 12 illustrates another embodiment of the current invention during a virtual examination where the physician have the patient to position to a silhouette for centering the screen.

### DETAILED DESCRIPTION OF THE INVENTION

Some embodiments are described in detail with reference to the related drawings. Additional embodiments, features, and/or advantages will become apparent from the ensuing description or may be learned by practicing the invention. The following description is not to be taken in a limiting sense, but is made merely for the purpose of describing the general principles of the invention. The steps described herein for performing methods form one embodiment of the invention, and, unless otherwise indicated, not all of the steps must necessarily be performed to practice the invention, nor must the steps necessarily be performed in the order listed. It should be noted that references to “an” or “one” or “some” embodiment(s) in this disclosure are not necessarily to the same embodiment, and such references mean at least one.

The present invention has been conceived with the aim at solving a crucial drawback of the current systems and methods that enable virtual medical office visits. The current systems do not provide for a palpation capability where a physician, despite of being remote, is aided by the patient and the current invention in palpating the area of concern to gain a complete diagnosis.

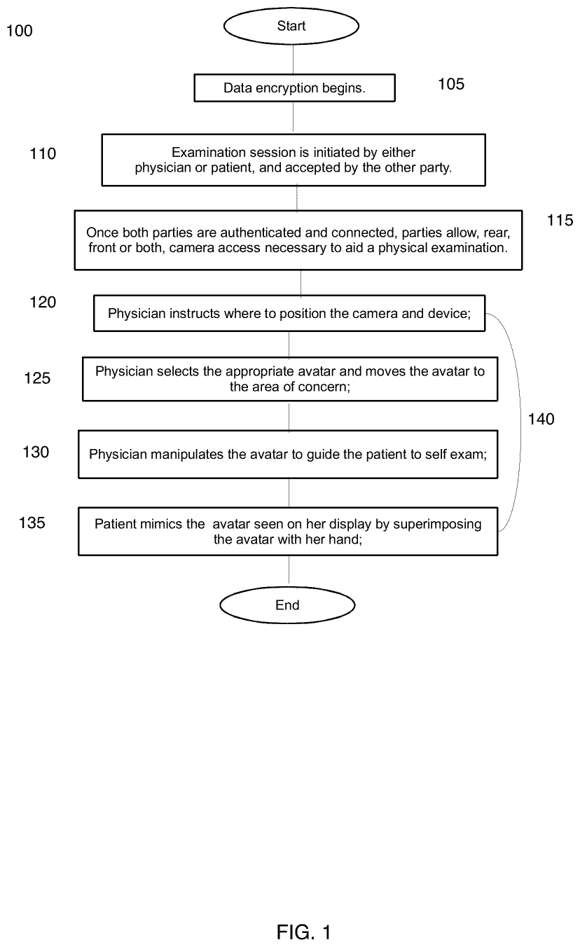

FIG. 1 presents a flowchart of an exemplary virtual examination. In this embodiment 100, a user of the system, either the patient or physician, initiates the virtual examination by launching the current system on a multimedia device. The current system, in turn, immediately initiates its security module which authenticates the parties accessing the system, encrypts all outgoing communication data and decrypts all incoming data. After logging in at step 105, the initiating party requests a communication session with another party via a session module at step 110. The session module manages the examination session and thus it sends an invitation to join to the other party. If the other party accepts this invitation, the party launches another instance of the current system on his or her device, logs in and accepts the invitation. Once the parties to the virtual examination are connected by the session module, they arrive at step 115 where they allow the current system access to the device's camera and microphone. At this point, the session module employs, among others, voice module, image module, and text module. The parties can allow rear, front, or both camera access depending on the examination need. After eliciting the problem or issue verbally, the physician then instructs the patient to move the device and camera to the area of concern at step 120. Alternatively, the patient just moves the camera to the area of concern to aid her description of the problem. The video images are transmitted from the patient's device to the physician's device where they are managed and displayed by the video module which, in turn, is managed by the session module. At this point, if needed, the physician selects an appropriate overlay, that is essentially digitally drawn likeness of a hand or objects, from a list of overlays shown by the current system on the physician's screen in step 125. The physician then superimposes his hand over the overlay, and makes necessary hand gestures, such as one or two-finger palpation, hand grabbing or squeezing posterior palpation, twisting, turning, pulling or pushing, or two-handed maneuvers, that are clinically used to physically exam the area of concern in step 130. As the physician hand gestures the physical examination, her front camera captures the movement and the current system's image module packages and transmits the movement to the patient's device. As it receives the image package, the patient's image module processes the package and controls a counterpart overlay on her screen to make the exact hand gestures. The patient then puts her hand on the area of concern such that the counterpart overlay superimposes her hand. Then, the patient just mimics the counterpart overlay's movement, and palpates the area of concern in step 135. As the examination needs, the physician can repeat the steps 120-135 for other areas of concern, different hand gestures, or examination tools other than hand, in the repetition 140. For a different hand overlay or examination tool choice, the physician selects the appropriate overlay shown on the screen and repeats the steps 130-135.

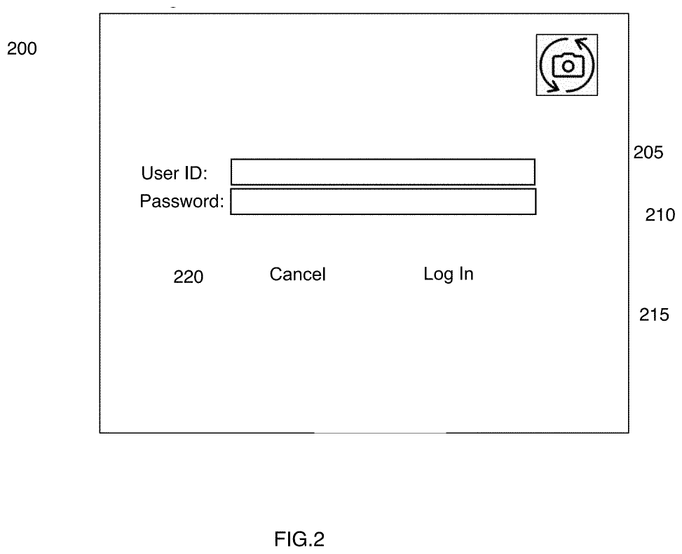

FIG. 2 illustrates an exemplary embodiment 200 of the current invention where it presents a login screen for the parties who want to access the current system. It is appreciated that other methods of authentication are contemplated, such as facial recognition, fingerprint scan, etc. The parties tap the text boxes, 205 and 210, adjacent to the labels “User ID” and “Password” to enter their pre-set user ID and password. Once the information is entered, the parties can tap the button 215, labeled “Log In”, to be authenticated and log in the system. If they want to cancel and exit the system, they can tap button 220, labeled “Cancel”, to do so.

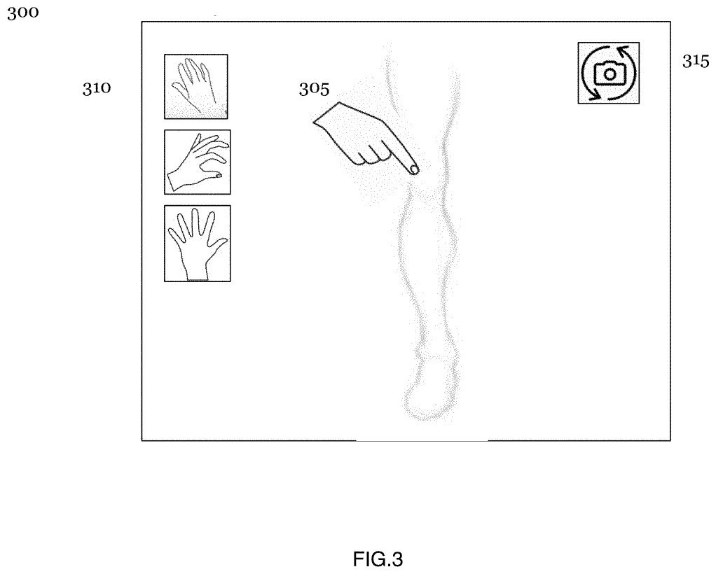

FIG. 3 illustrates another exemplary embodiment 300 of the current invention where the main screen of the current system is shown running on the physician's device. The main display shows the patient's leg and the knee seems the area of concern on where a hand overlay is pointing guided by the physician's hand. In this instance, the physician guides the hand overlay by moving his finger over his own electronic device. It is appreciated that the hand overlay 305 is one of many different hand overlay as well as tools (not shown) used for the examination. The overlay collection 310 allows the physician to select the appropriate tool. It is appreciated that the collection 310 can include many more overlays of any shape, e.g., hand, tool, arrow, or device, It is anticipated that the system will develop a library of tool overlays for all examination purposes.

Certainly, the physician can switch among the overlays anytime during a virtual physical examination, depending on what physical examination maneuver he wants to perform at the time. Furthermore, although the hand overlay is shown translucent here, the physician can change it to an opaque overlay. In addition to the overlay collection 310, the physician can also switch among different modes of camera (front, rear, or both) by tapping on the camera image button 315, and cycling through the modes until the right mode is present. The patient also has a similar camera image button on her screen so she can select the appropriate camera mode for the examination purpose at the moment. It is also contemplated that the physician can use and select more than more than one overlay if the examination requires. For example, the physician performs a more complex two-handed maneuver twisting an anatomical landmark across two hands with one hand shifting anatomical landmark medially, laterally, superiorly, or inferiorly. Other complex hand maneuvers include percussing, open hand palpation or squeezing, twisting, pushing down, pulling up, pushing in, pulling out, anterior pulling, posterior pulling, anterior pushing, posterior pushing, pushing laterally, medially, superiorly, or inferiorly, grabbing and moving laterally, medially, superiorly, inferiorly, and rotating, etc.

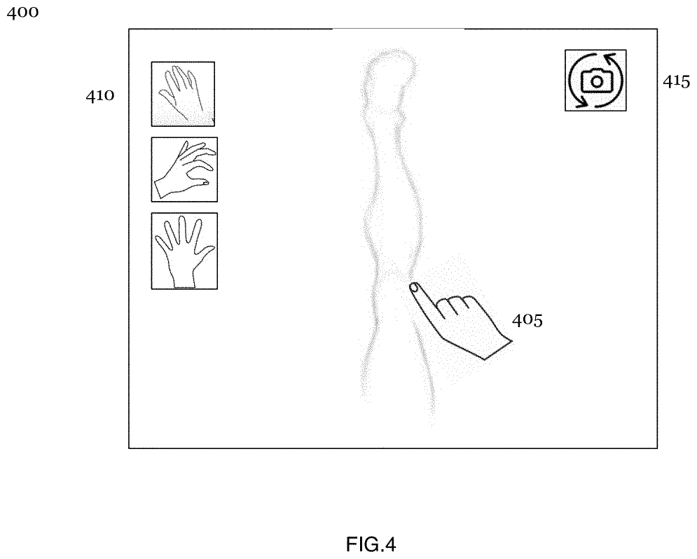

FIG. 4 illustrates an exemplary embodiment 400 of the current invention running on the patient's device, as a counterpart of the physician's. The shown hand overlay 405 is remotely controlled by the physician. As the physician, described in FIG. 3, moves his finger to point at the knee, the counterpart overlay 405 on the patient's screen moves and points at the knee. It is appreciated that the overlays' movement can be bi-directional; that is, the patient can also move the overlay to accurately show the physician the area of concern. Similarly, the patient can also selection different overlays from the overlay collection 410. However, the patient's collection 410 includes a different set of overlays. The patient can also select the right camera mode for the current examination purpose by tapping the camera image button 415 and cycling through the modes. When the physician wants the patient to palpate the area of concern, the physician directs the patient to place her hand or a medical tool on the area of concern such that the overlay superimposes the hand or tool. The patient then mimics the overlay's movement, which is created by the physician remotely, to perform the examination maneuver. It is appreciated that the physician can move the overlay over various anatomic landmarks sequentially to allow physical examination of different anatomic landmarks.

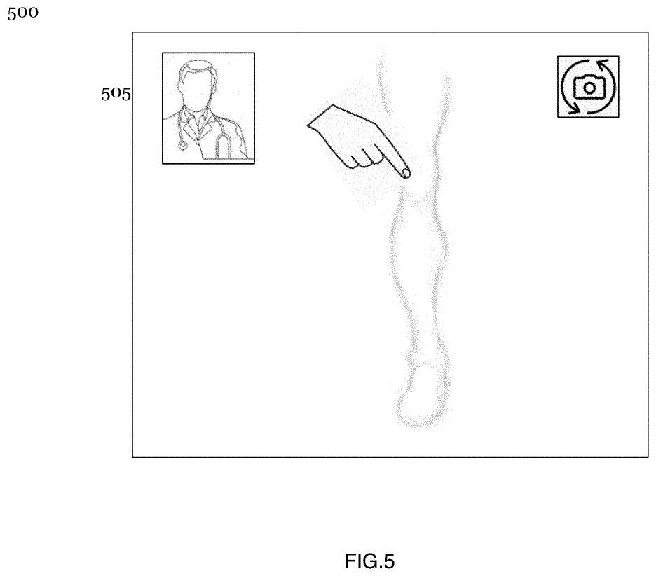

FIG. 5 illustrates another exemplary embodiment 500 of the current invention running on the patient's device. This embodiment further comprises a picture-in-picture window 505 that allows the patient to see the physician on the screen. Similarly, the physician can see the patient on his device screen in a picture-in-picture window. Observing the patient's facial expression that reflects a pain sensation while examining the area of concern is crucial in some situations. digital device to provide feedback data to a digital device.

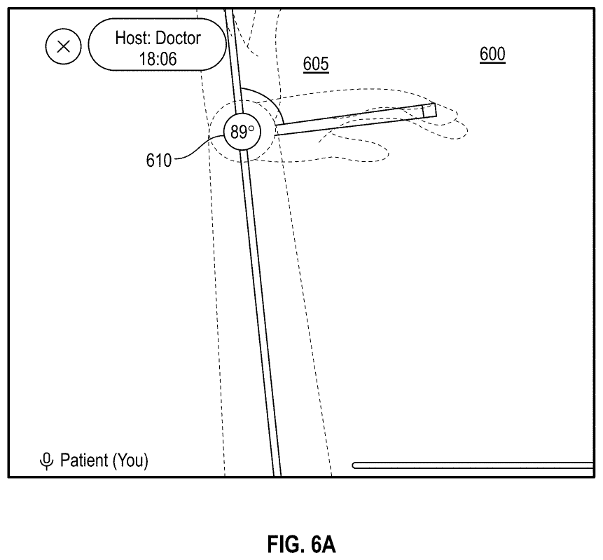

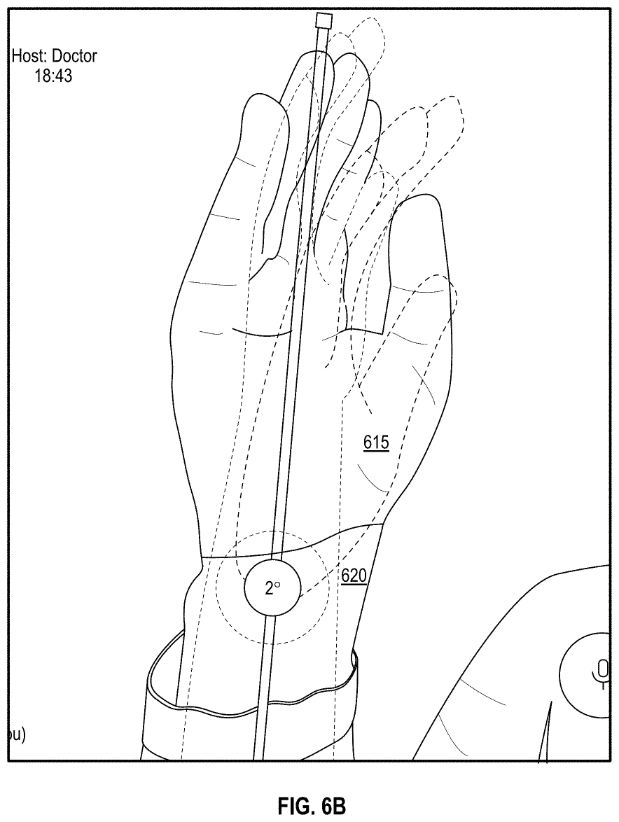

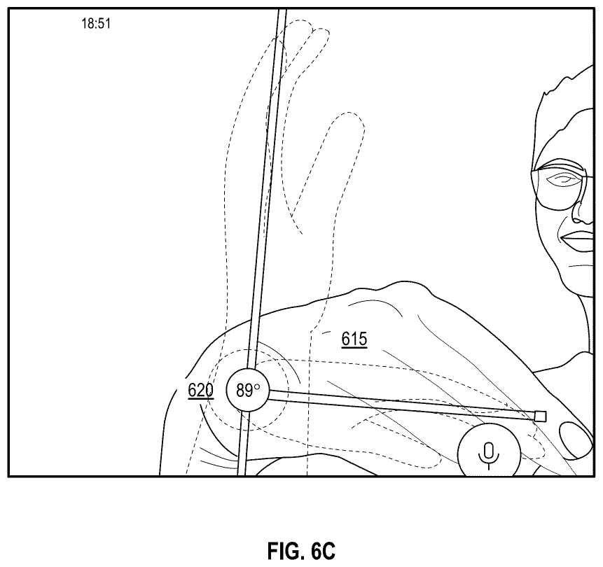

FIGS. 6A, 6B, and 6C show a wrist examination using an embodiment 600 of the current invention. FIG. 6A shows the physician's screen where he selects a hand overlay 605 and places it in the proximity of the patient's wrist. As described above, the patient's hand and wrist images are captured remotely by the patient's device, transmitted to and shown on the physician's device. The physician further selects a goniometer overlay 610 and placed it on top of the hand overlay. The goniometer provides a precise angle of a hand gesture. In this instance, the physician wants to instruct the patient to bend her wrist as shown by a 89 degree angle.

FIG. 6B shows the patient's screen with the counterpart overlays, hand overlay 615 and goniometer overlay 620, which superimpose the patient's hand. When the hand overlay 605 on the physician's screen is made to move by the physician, the counterpart hand overlay 615 on the patient's screen moves in tandem. Before doing the hand gesture, the physician instructs the patient through the session module and voice module at the other end of the session to put her hand such that the hand overlay 615 superimposes her hand. Then as the hand overlay 615 moves, the patient mimics the gesture by bending her hand to match the hand overlay as shown in FIG. 6C. The goniometer overlay helps the physician and patient to bend the hand and wrist at a precise angle.

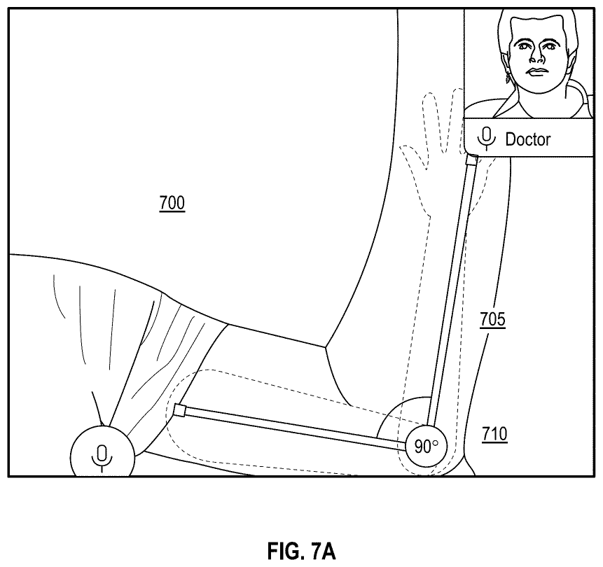

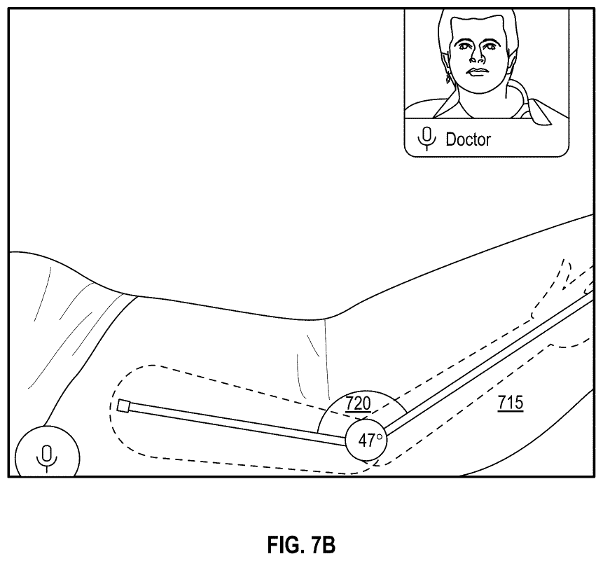

FIGS. 7A and 7B show an elbow examination using an embodiment 700 of the current invention. FIG. 7A shows the physician's screen with the patient's arm and elbow image being transmitted from the patient's device cameras through the image modules and session modules at both ends of the session, and being displayed on the physician's screen. The physician selects the arm and goniometer overlays, 705 and 710, respectively, to show the patient a bent arm overlay 705 at the elbow at a precise 90 degree angle. The patient adjusts the angle of her arm at the other end to match the angle shown by the counterpart arm and goniometer overlays, 715 and 720, as illustrated in FIG. 7B. The physician then moves the lower part of the arm overlay 705 by dragging the part with the physician's finger on the screen at the desired angle, which is 137 degree in this instance. At the other end of the session, the patient matches the movement with her arm after putting the arm overlay to superimpose her arm as shown in FIG. 7B.

As disclosed, the current invention allows more accurate live report and observation by providing a physician a capability of indirect palpating close or obstructed anatomic landmarks, such as a knee bone or abdomen. Similarly, pain sensation is easier gauged where the physician can observe the patient's facial expression while palpating the pain site, as opposed to verbal description alone. Thus, the current invention improves the accuracy and efficiency in sorting through differential diagnoses.

It is further contemplated that the current invention can be installed and run on multiple devices at the physician's or patient's location. The system can synchronize multiple screens of the devices to show various angles of an area of concern. In another embodiment, the patient can capture various angles of the area to be examined, the system displays the captured images side by side, and the physician places overlays over the images to show a complicated examination technique. This capability can be used to record a problem where live communication is not possible or available. The physician then uses this capability to record a response. Similarly, the system also allows the patient to record a video showing her medical issue and send it to the physician. In response, the physician can record a instructional multimedia video response using various overlays to show the patient the examination techniques, and forward it to the patient so that the patient can self-exam.

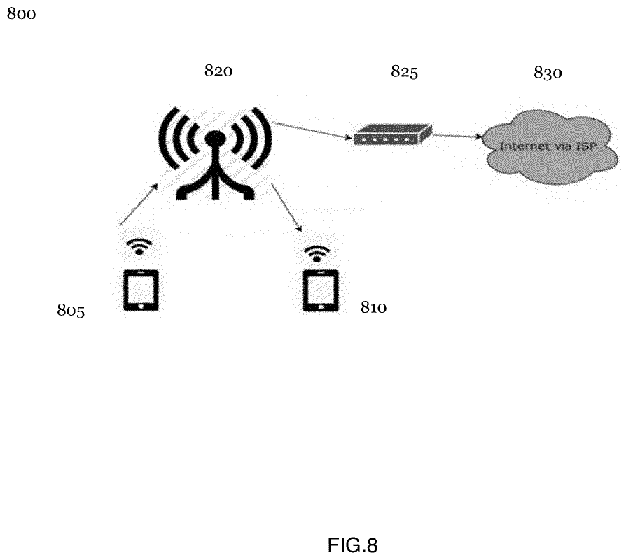

FIG. 8 illustrates an exemplary network system 800 of the current invention. The initiating party's device and session module 805 establishes a communication channel with a signal transmitting tower 820 that can be a LAN or broadband tower. This tower 820 then transmits the communication signal to a router 825 that then transmits the signal through the Internet 830. At the other end of the communication channel, after accepting the invitation to join the session, the participating party's device and session module 810 will receive and send communication signal through a transmitting tower, a router, and the Internet. The communication signal can carry voice, image, and text data simultaneously.

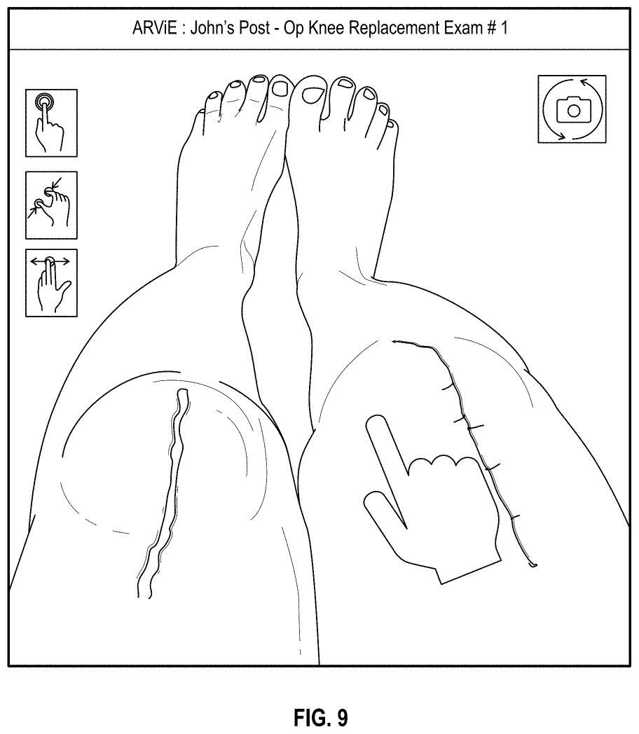

FIG. 9 shows a knee examination using an embodiment 900 of the current invention. FIG. 9 shows the physician's screen where he selects a hand overlay 904 and places it in the proximity of the patient's knee. As described above, the patient's knee images are captured remotely by the patient's device, transmitted to and shown on the physician's device. The physician further can select between the pinching overlay 901, squeezing 902 overlay, and palpation overlay 903, and placed it on top of the knee. While pinching overlay 901 signifies for patient to carry out the act of punching nearing the healing area 905, the squeezing overlay 902 instructs patient to squeeze and the palpation overlay 903 instructs the patient to carry out the act of palpation near the healing area 905. In the user interface, window 907 provides for either activation to switch between the view to the treatment area or the face of the doctor or the face of the patient or allowing the face of the doctor or patient to be shown concurrently with the screen 900.

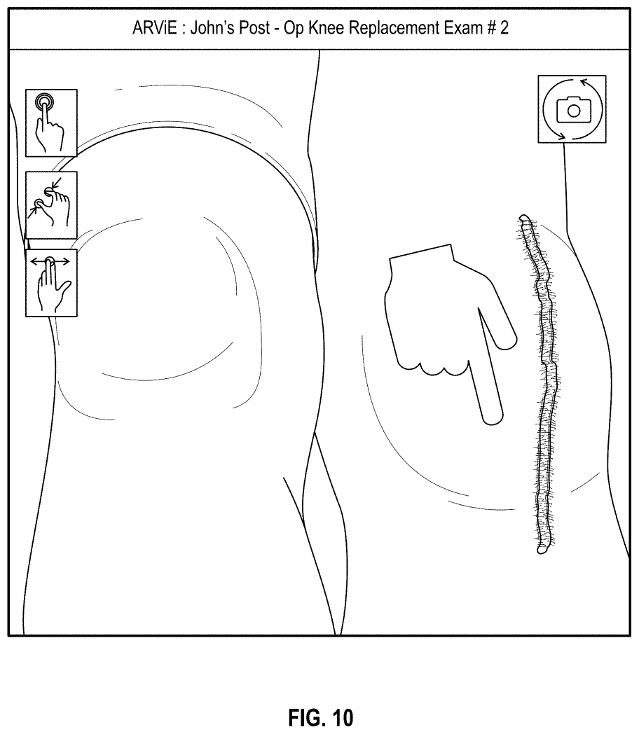

FIG. 10 shows a knee examination using an embodiment 1000 of the current invention. Unlike Screen 900, FIG. 10 shows a more zoomed in focus on the scar area 1006 and the still the physician's screen allows physician to select a hand overlay 1004 and places it in the proximity of the patient's knee. As described above, the patient's knee images are captured remotely by the patient's device, transmitted to and shown on the physician's device. The physician further can select between the pinching overlay 1001, squeezing 1002 overlay, and palpation overlay 1003, and placed it on top of the knee. While pinching overlay 1001 signifies for patient to carry out the act of punching nearing the healing area 1005, the squeezing overlay 1002 instructs patient to squeeze and the palpation overlay 1003 instructs the patient to carry out the act of palpation near the healing area 1005. In the user interface, window 1007 provides for either activation to switch between the view to the treatment area or the face of the doctor or the face of the patient or allowing the face of the doctor or patient to be shown concurrently with the screen 1000.

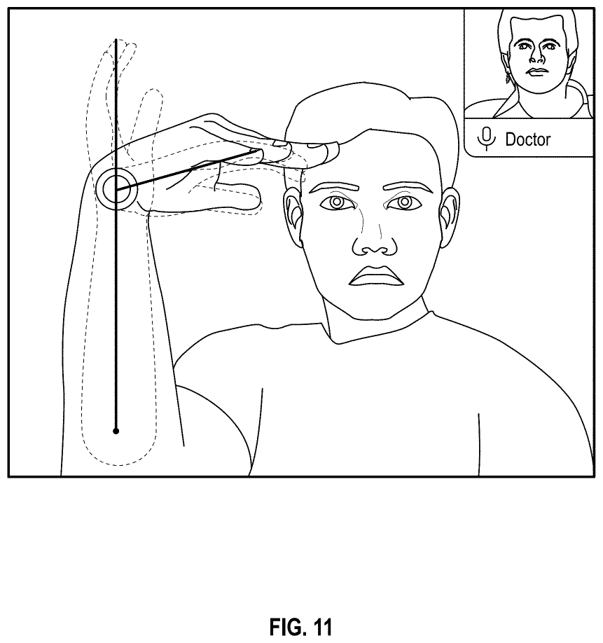

FIG. 11 shows a wrist examination using an embodiment 1100 of the current invention. FIG. 11 shows the physician's screen he can see the entire body 1102 and face 1101 of the patient while performing the AR examination as oppose to screen in FIG. 6A is zoomed to focused on the treatment area. The choice of zoomed in and out is function of the user interface. It is also the case the screen 1103 which shows the face of the doctor both in the screen of the patient and the doctor.

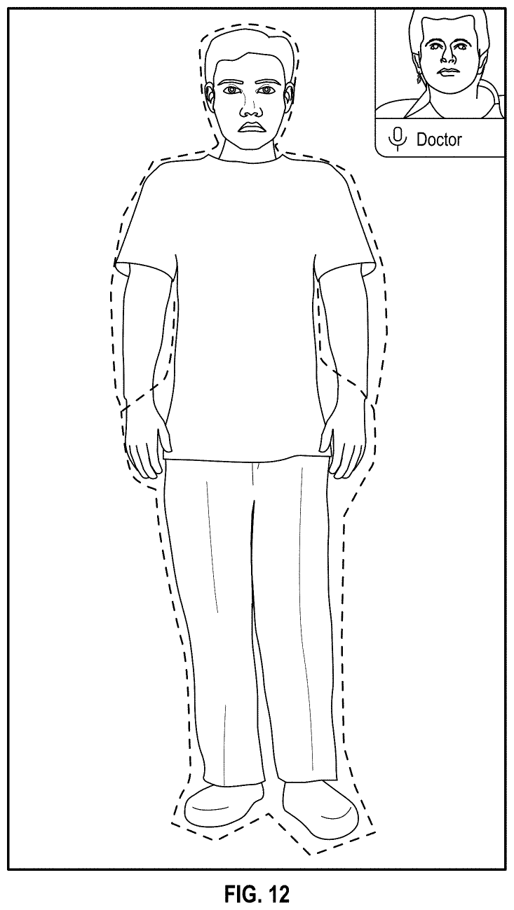

FIG. 12 shows the setup screen 1200 in which a patient 1201 is asked to be positioned within the silhouette overlay 1202 where the patient can position the mobile device within the arm's reach length and be centered in the setup screen 1200 so the examination using overlay can initiate. Similarly, there is screen on the upper right hand corner allowing for the face of either the patient and or doctor.

The invention claimed is:

1. A system for remote examination of patient by a doctor, said system comprising:
a. a doctor mobile computer device comprising a first set of processors, said first set of processors executing an application embedded within said doctor mobile computer device;
b. a patient mobile computer device comprising a second set of processors, said second set of processors executing said application embedded within said patient mobile computer device;
c. said application comprising:
A. a camera module wherein said module captures a video feed of an area of a body part of said patient and sends said video feed to a patient viewing module and a doctor view module to display said area of body part;
B. said patient viewing module displaying said video feed;
C. said doctor viewing module displaying said video feed and a plurality of inspection gesture buttons comprising a one-finger palpation gesture button, a two-finger palpation gesture button, a hand grabbing gesture button, a posterior palpation gesture button and a plurality of unique gesture graphics corresponding to said inspection gesture buttons;
D. a control module wherein said control module allows said doctor to select one of said inspection gesture buttons and one of said unique gesture graphics superimpose onto said area of said body part of said video feed wherein said control module further allows said doctor to move said unique gesture graphic within said area of said video feed as said unique gesture graphic continues to superimpose onto said area of said body part.

2. The system of claim 1 wherein said plurality of inspection gesture buttons further comprising a twisting gesture button.

3. The system of claim 1 wherein said plurality of inspection gesture buttons further comprising a pulling gesture button.

4. The system of claim 1 wherein said plurality of inspection gesture buttons further comprising a turning gesture button.

5. The system of claim 1 wherein said plurality of inspection gesture buttons further comprising a pushing up gesture button.

6. The system of claim 1 wherein said plurality of inspection gesture buttons further comprising a pushing down gesture button.

7. The system of claim 1 wherein said plurality of inspection gesture buttons further comprising a two-handed maneuvers gesture button.

8. The system of claim 1 wherein said plurality of inspection gesture buttons further comprising a two-handed maneuvers gesture button.

9. The system of claim 1 wherein said plurality of inspection gesture buttons further comprising a 2 hands Lachman's gesture button.

10. The system of claim 1 wherein said plurality of inspection gesture buttons further comprising a grabbing gesture button.

11. The system of claim 1 wherein

12. A system for remote examination of patient by a doctor, said system comprising:
a. a doctor mobile computer device comprising a first set of processors, said first set of processors executing an application embedded within said doctor mobile computer device;
b. a patient mobile computer device comprising a second set of processors, said second set of processors executing said application embedded within said patient mobile computer device;
c. said application comprising:
A. a camera module wherein said module captures a video feed of an area of a body part of said patient and sends said video feed to a patient viewing module and a doctor view module to display said area of body part;
B. said patient viewing module displaying said video feed;
C. said doctor viewing module displaying said video feed and a examination icon comprising a first body part overlay and a second part body part and a first goniometer overlay and a second goniometer overlay wherein said first body part overlay encloses said first goniometer overlay and said second body part overlay encloses said second goniometer overlay such that when first body part moves in relation to said second body part, said first goniometer overlay moves in relation to said second goniometer overlay and an angular degree display measuring the angular degree between said first goniometer overlay and said second goniometer overlay;
D. a control module wherein said control module superimposes said examination icon onto said area of said body part of said video feed wherein said control module further allows said doctor to move said examination icon within said area of said video feed as said examination icon continues to superimpose onto said area of said body part.

13. The system of claim 12, wherein said control module further allows said doctor to move said first body part overlay in relation to second body part overlay as said first body part overlay, said second body part overlay, said first goniometer overlay, said second goniometer overlay, and said angular degree display continue to superimpose onto said area of said body part.
